(19) United States
(12) Patent Application Publication
(10) Pub. No.: US 2024/0415570 A1
(43) Pub. Date: Dec. 19, 2024

(54) DEVICES, SYSTEMS, AND METHODS FOR PREVENTING ARCING BETWEEN ELECTRODES FOR MEDICAL PROCEDURES

(71) Applicant: BOSTON SCIENTIFIC SCIMED, INC., MAPLE GROVE, MN (US)

(72) Inventors: Laura Emily Sabban, Plainville, MA (US); Timothy A. Ostroot, Cokato, MN (US); Natalie White, Marlborough, MA (US); James J. Scutti, Norwell, MA (US)

(73) Assignee: BOSTON SCIENTIFIC SCIMED, INC., MAPLE GROVE, MN (US)

(21) Appl. No.: 18/740,868

(22) Filed: Jun. 12, 2024

Related U.S. Application Data
(60) Provisional application No. 63/472,711, filed on Jun. 13, 2023.

(51) Int. Cl. A61B 18/14; A61B 18/00; A61B 90/00

(52) U.S. Cl. CPC A61B 18/1492; A61B 2018/00083; A61B 2018/0016; A61B 2018/00577; A61B 2090/3925

(57) ABSTRACT

An energy-delivering assembly probes formed from an elongated energy-delivering member have one or more electrodes defined along an energy-delivering region of the energy-delivering member. An electrode-defining insulation member is provided over the energy-delivering member to define and space apart at least a first electrode from a second electrode. One or more of the electrodes and/or insulation members are configured and/or have characteristics or properties which reduce/minimize/eliminate arcing between/across electrodes of the energy-delivering assembly.

### CROSS-REFERENCE TO RELATED APPLICATIONS

This application claims the benefit of priority under 35 U.S.C. § 119 to U.S. Provisional Application No. 63/472,711, filed Jun. 13, 2023, the entire disclosure of which is hereby incorporated by reference herein for all purposes.

### FIELD

The present disclosure relates generally to the field of medical devices, assemblies, systems, and methods used in applying energy to a patient such as for therapeutic purposes. More particularly, the present disclosure relates to the field of medical devices, assemblies, systems, and methods for applying electrical energy, such as therapeutic electrical pulses, to a patient. Even more particularly, the present disclosure relates to various devices, assemblies, systems, and methods for electroporation treatment. And, even more particularly, the present disclosure relates to bipolar devices, assemblies, and systems applying energy, such as electrical energy such as for electroporation, and associated methods.

### BACKGROUND

Various devices, assemblies, systems, and methods exist for energy-based medical treatment and/or therapeutic protocols. For instance, various focal therapy devices are configured to apply energy to debulk target tissue or to eliminate malignant cells. Various technologies for such therapy rely on thermal effects, such as radiofrequency (“RF”) heating, microwave heating, cryoablation, high intensity focused ultrasound (“HIFU”), etc. In contrast, electroporation and/or irreversible electroporation is a non-thermal therapy, and has significant potential benefits over thermal modalities. Energy may be applied to perform electroporation and/or irreversible electroporation (“IRE”) as a mode of treating various conditions and/or diseases using an electric field to interrupt and/or to change the nature of biological cellular matter. For instance, the applied electric field may significantly increase the electrical conductivity and permeability of the plasma in the cell membrane. The applied energy causes paths/pores to open within cell walls and/or membranes near the device applying the energy (e.g., near the electrode, probe, etc., thereof). The electric field disrupts homeostasis, and, in the case of IRE, kills the cells, such as through apoptosis and/or necrosis. Development of this technology has led to the creation of bipolar probes (with two electrodes, typically separated by insulation) to mitigate certain effects from exposure to electrical fields (e.g., muscle stimulation such as which may result in muscle contractions; cardiac interference, etc.). Once positioned within the target site (e.g., a tumor), the device is activated, such as by generating an electric field between and/or around the electrodes of the device, for various therapeutic procedures. One challenge with bipolar probes is the potential for arcing to occur from one electrode to the other. Arcing can adversely affect treatment efficiency as well as efficacy. There remains a need for at least reduction or mitigation of the formation of unintended or undesired electric field concentrations. Furthermore, there remains a need for reducing, and preferably preventing, arcing from occurring.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an energy-delivering assembly includes an energy-delivering member formed of an electrically-conductive material; an electrode-defining insulation member positioned over a portion of the energy-delivering member to define a first electrode spaced apart from a second electrode by the electrode-defining insulation member; and at least one arc-reducing interface between the electrode-defining insulation member and an adjacent one of the first and second electrodes.

In some aspects, the arc-reducing interface comprises a region comprises a modified shape of at least one of the electrode-defining insulation member or the adjacent one of the first and second electrodes. In some aspects, the arc-reducing interface is in the form of a chamfer or fillet along an interface between the electrode-defining insulation member and the adjacent one of the first and second electrodes. Additionally or alternatively, in some aspects, the arc-reducing interface is in the form of a rolled wall of the first electrode adjacent the electrode-defining insulation member. In some aspects, the arc-reducing interface is in the form of a reduction in thickness in the electrode-defining insulation member adjacent to the adjacent one of the first and second electrodes relative to an intermediate region of the electrode-defining insulation member spaced apart from the adjacent one of the first and second electrodes. Additionally or alternatively, in some aspects, the arc-reducing interface is in the form of discontinuities in the electrode-defining insulation member at least adjacent to the adjacent one of the first and second electrodes. In some aspects, the electrode-defining insulation member is slotted to define the discontinuities therealong.

Additionally or alternatively, at least one of the first electrode and the second electrode has an end adjacent the electrode-defining insulation member and an end spaced away from the electrode-defining insulation member; echogenic features are provided closer to the end of the at least one of the first electrode and the second electrode spaced away from the electrode-defining insulation member than the end of the at least one of the first electrode and the second electrode adjacent the electrode-defining insulation member; and the end of the at least one of the first electrode and the second electrode adjacent the electrode-defining insulation member and without echogenic features defines the arc-reducing interface.

Additionally or alternatively, the arc-reducing interface is formed by doping at least a region of the electrode-defining insulation member to create a gradient in electrical conductivity between the electrode-defining insulation member and the adjacent one of the first and second electrodes.

Additionally or alternatively, the arc-reducing interface includes a non-insulating additional member between the electrode-defining insulation member and the adjacent one of the first and second electrodes, and having an electrical conductivity less than that of the adjacent one of the first and second electrodes.

Additionally or alternatively, the arc-reducing interface includes a coating over the adjacent one of the first and second electrodes.

Additionally or alternatively, the first electrode and the second electrode are colinear to define the energy-delivering assembly as a bipolar linear probe.

In accordance with various principles of the present disclosure, an energy-delivering treatment system includes an energy-delivering assembly including an energy-delivering member formed of an electrically-conductive material; an electrode-defining insulation member positioned over a portion of the energy-delivering member to define a first electrode spaced apart from a second electrode by the electrode-defining insulation member; and at least one arc-reducing interface between the electrode-defining insulation member and an adjacent one of the first and second electrodes. The system further includes a power connector configured to deliver energy to the energy-delivering assembly to deliver energy to the energy-delivering member to deliver energy along the electrodes thereof.

In some aspects, the arc-reducing interface includes a region comprises a modified shape of at least one of the electrode-defining insulation member or the adjacent one of the first and second electrodes.

Additionally or alternatively, at least one of the first electrode and the second electrode has an end adjacent the electrode-defining insulation member and an end spaced away from the electrode-defining insulation member; echogenic features are provided closer to the end of the at least one of the first electrode and the second electrode spaced away from the electrode-defining insulation member than the end of the at least one of the first electrode and the second electrode adjacent the electrode-defining insulation member; and the end of the at least one of the first electrode and the second electrode adjacent the electrode-defining insulation member and without echogenic features defines the arc-reducing interface.

Additionally or alternatively, the arc-reducing interface is formed by doping at least a region of the electrode-defining insulation member to create a gradient in electrical conductivity between the electrode-defining insulation member and the adjacent one of the first and second electrodes.

Additionally or alternatively, the arc-reducing interface includes a non-insulating additional member between the electrode-defining insulation member and the adjacent one of the first and second electrodes, and having an electrical conductivity less than that of the adjacent one of the first and second electrodes.

Additionally or alternatively, the arc-reducing interface includes a coating over the adjacent one of the first and second electrodes.

In accordance with various principles of the present disclosure, a method of applying electroporation or irreversible electroporation energy includes use of a multi-electrode energy-delivering treatment system having at least a first electrode and a second electrode defined along an energy-delivering member of the multi-electrode energy-delivering treatment system by an electrode-defining insulation member positioned over the energy-delivering member to define and space apart the first electrode and the second electrode from each other. In some aspects, the method includes delivering energy from an energy source to the energy-delivering member of the multi-electrode energy-delivering treatment system to deliver electroporation or irreversible electroporation energy to the first electrode and the second electrode of the multi-electrode energy-delivering treatment system; and creating a gradient in electrical conductivity between the electrode-defining insulation member and at least one of the first electrode or the second electrode of the multi-electrode energy-delivering treatment system to reduce arcing between the first electrode and the second electrode thereof.

In some aspects, the method includes creating a gradient in electrical conductivity by at least one of: modifying features of at least one of the first electrode or the second electrode of the multi-electrode energy-delivering system; modifying features of the electrode-defining insulation member; adding a non-insulating member between the electrode-defining insulation member and at least one of the first electrode or the second electrode, the non-insulating member having an intermediate electrical conductivity less than the electrical conductivity of the least one of the first electrode or the second electrode; or coating at least a portion of at least one of the first electrode and the second electrode.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing by a multiple of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of an example of an embodiment of an energy-delivering treatment system formed in accordance with aspects of the present disclosure.

FIG. 1A illustrates a detail view along detail area 1A in FIG. 1 illustrating further details of an example of an embodiment of an energy-delivering assembly formed in accordance with various principles of the present disclosure and usable in an energy-delivering treatment system as illustrated in FIG. 1.

FIG. 2 illustrates an elevational view of an embodiment of an energy-delivering treatment system formed in accordance with aspects of the present disclosure to reduce/minimize/eliminate arcing between electrodes thereof such as via modified echogenic features.

FIG. 3 illustrates an elevational view of an embodiment of an energy-delivering treatment system formed in accordance with aspects of the present disclosure to reduce/minimize/eliminate arcing between electrodes thereof such as via modified interfaces between an electrode and an insulation member thereof.

FIG. 4 illustrates an elevational and partially cross-sectional view of an embodiment of an energy-delivering treatment system formed in accordance with aspects of the present disclosure to reduce/minimize/eliminate arcing between electrodes thereof such as via one or more modified interfaces between an electrode and an insulation member thereof.

FIG. 5 illustrates an elevational view of an embodiment of an energy-delivering treatment system formed in accordance with aspects of the present disclosure to reduce/minimize/eliminate arcing between electrodes thereof such as via a modified insulation member between electrodes thereof.

FIG. 6 illustrates an elevational view of an embodiment of an energy-delivering treatment system formed in accordance with aspects of the present disclosure to reduce/minimize/eliminate arcing between electrodes thereof such as via a modified insulation member between electrodes thereof.

FIG. 7 illustrates an elevational view of an embodiment of an energy-delivering treatment system formed in accordance with aspects of the present disclosure to reduce/minimize/eliminate arcing between electrodes thereof such as via modified material between at least one electrode and insulation member thereof.

FIG. 8 illustrates an elevational view of an embodiment of an energy-delivering treatment system formed in accordance with aspects of the present disclosure to reduce/minimize/eliminate arcing between electrodes thereof such as via modified material between at least one electrode and insulation member thereof.

FIG. 9 illustrates an elevational view of an embodiment of an energy-delivering treatment system formed in accordance with aspects of the present disclosure to reduce/minimize/eliminate arcing between electrodes thereof such as via an intermediate material between at least one electrode and insulation member thereof.

FIG. 10 illustrates an elevational view of an embodiment of an energy-delivering treatment system formed in accordance with aspects of the present disclosure to reduce/minimize/eliminate arcing between electrodes thereof such as via at least one modified electrode thereof.

FIG. 11 illustrates an elevational view of an embodiment of an energy-delivering treatment system formed in accordance with aspects of the present disclosure to reduce/minimize/eliminate arcing between electrodes thereof such as via at least one modified electrode thereof.

FIG. 12 illustrates an elevational view of an embodiment of an energy-delivering treatment system formed in accordance with aspects of the present disclosure to reduce/minimize/eliminate arcing between electrodes thereof such as via additional modifications to at least one modified electrode thereof.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As understood herein, corresponding is intended to convey a relationship between components, parts, elements, etc., configured to interact with or to have another intended relationship with one another. As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. For the sake of convenience, reference may be made to terms such as therapy, treatment, diagnosis, procedure, etc., including various grammatical forms thereof, alternately and without intent to limit, reference to one such term not excluding the others unless explicitly stated. Moreover, it will be appreciated that reference may be made herein to a treatment site, target site, site, etc., interchangeably and without intent to limit. Finally, reference to “at” a location or site is intended to include at and/or about the vicinity of (e.g., along, adjacent, proximate, etc.) such location or site.

As generally used herein, the term “ablation” generally refers to removal of cells either directly or indirectly by supply of energy within an electric field and may include removal by loss of cell function, cell lysis, coagulation, protein denaturation, necrosis, apoptosis, and/or irreversible electroporation. “Ablation” may similarly refer to creation of a lesion by ablation. Additionally, the terms “undesirable tissue,” “target cells,” “diseased tissue,” “diseased cells,” “tumor,” “cell mass” may be used herein to refer to cells removed or to be removed, in whole or in part, by ablation, and are not intended to limit application of any assemblies, systems, devices, or methods described herein. For example, such terms include ablation of both diseased cells and certain surrounding cells, despite no definite indication that such surrounding cells are diseased. Ablation performed by assemblies, systems, devices, or methods described herein may be of cells located around a biological lumen, such as a vascular, ductal, or tract area, for example, to create a margin for a medical professional to resect additional cells by ablation or other method. In accordance with various principles of the present disclosure, devices, assemblies, systems, and methods disclosed herein may be configured for performing ablation via electroporation and/or IRE.

In certain embodiments, electrical ablation devices may generally comprise one or more electrodes configured to be positioned into or proximal to undesirable tissue in a tissue treatment region (e.g., a target site or a worksite). The tissue treatment region may have evidence of abnormal tissue growth. In general, the electrodes may include an electrically conductive portion and may be configured to be electrically coupled to an energy source. Once the electrodes are positioned into or across (e.g., extending beyond a tumor/mass on both sides) to the undesirable tissue, an energizing potential may be applied to the electrodes to create an electric field to which the undesirable tissue is exposed. The energizing potential (and the resulting electric field) may be characterized by various parameters, such as, for example, frequency, amplitude, pulse width (duration of a pulse or pulse length), and/or polarity. Suitable energy sources include electrical waveform generators, such as waveform generators capable of creating IRE, high frequency IRE, NanoPulse, and/or ablative waveforms. The energy source generates an electric field with desired characteristics for the treatment to be performed at the target site, such as based on the treatment site, application, device, etc. For instance, the electric field may be generated to have suitable characteristic waveform output in terms of voltage, impedance, frequency, amplitude, pulse width, delays (e.g., delays between pulses), number of pulses per burst, number of bursts, and waveform polarity (monopolar vs. bipolar). The electric current flows between the electrodes and through the tissue based on the applied potential and tissue impedance. The supplied electric current provided by the energy source may deliver a pulse sequence to the target site. For example, an energy source may supply various waveforms in one or more pulse sequences tailored to the desired application

In some aspects, the devices, assemblies, systems, and methods of the present disclosure are configured for use in electroporation and/or irreversible electroporation (“IRE”) treatments/therapies. For instance, devices, assemblies, systems, and methods may be configured in accordance with various principles of the present disclosure for minimally invasive ablation treatment of undesirable tissue through the use of IRE. Minimally invasive ablation treatment may be characterized by the ability to ablate selected tissue in a controlled and focused manner with reduced or no thermally-damaging effects to surrounding healthy tissue.

In accordance with various principles of the present disclosure, an energy-delivering treatment system capable of performing electroporation and/or IRE includes an electrically-conductive elongate body defining first and second electrode portions therealong, such as to form a bipolar probe. More particularly, the electrode portions of the electrically conductive elongate body may be formed of an electrically conductive material such as medical grade stainless steel, platinum, gold, nitinol, a cobalt-chromium alloy, a nickel-cobalt alloy such as MP35N, or other alloys, or materials plated with electrically-conductive materials, etc. An insulation member is positioned between the electrodes, such as to electrically isolate the electrodes (typically with one electrode serving as an anode and another electrode serving as a cathode). To effectively treat a target site with electroporation and/or IRE, an effective electric field must be created between the two electrodes of the bipolar probe. However, application of energy with bipolar probes may be accompanied by arcing of energy between the electrodes of the probe. In particular, the risk of arcing increases the closer the electrodes are to each other and/or the higher the voltage across the electrodes of the probe and/or the higher the current applied to the probe. Typically, the smaller the target site for treatment (e.g., the smaller the tumor to be treated/ablated/otherwise affected by the electroporation and/or IRE energy), the higher the possibility of arcing between the electrodes.

In accordance with various principles of the present disclosure, a multi-electrode energy-delivering assembly, such as a bipolar probe, is configured to reduce arcing between the electrodes thereof. More particularly, various features between the electrodes of a multi-electrode energy-delivering assembly formed in accordance with various principles of the present disclosure are configured to reduce the likelihood of arcing and thereby to increase effectiveness and efficiency of the assembly. In some aspects, the multi-electrode energy-delivering assembly is a bipolar probe. In some aspects, the bipolar probe is a linear bipolar probe, with a first electrode separated from a second electrode by an insulation member. In accordance with various principles of the present disclosure, one or more interfaces between the electrodes of the energy-delivering assembly, such as one or more interfaces between an electrode and an adjacent insulation member, are modified to reduce, if not eliminate, the likelihood of arcing. The modifications may include one or more of the following: modifications to features of one or both of the electrodes, such as echogenic features, and/or sizes, shapes, configurations, properties (e.g., conductivities), and/or dimensions of one or both of the electrodes; modifications to the size, shape, configuration, properties (e.g., conductivity), and/or dimensions (e.g., thickness) of an insulation member defining and/or spacing apart electrodes; addition of materials between electrodes and an insulation member defining and/or spacing apart electrodes, and/or over one or more electrodes. For the sake of convenience, and without intent to limit, interfaces modified in accordance with various principles of the present disclosure to reduce arcing may be generically referenced herein as arcing-reducing interfaces.

The energy-delivering assembly may be deliverable through an elongate tubular member (e.g., a delivery sheath, catheter, working channel of an endoscope, etc.) inserted into a patient (such as through a natural anatomical passage or orifice and into a body lumen within a patient), or transcutaneously or percutaneously. The energy-delivering member of the energy-delivering assembly may be coupled to an energy source to energize the electrode portion thereof to apply an electric current to biological tissue. The energy source may be operative to generate an electric field between the electrode portion and another electrode portion, such as an electrode portion coupled to the energy source and having an opposite polarity, e.g., a return or ground. Once an energy-delivering assembly formed in accordance with various principles of the present disclosure is positioned at or near undesirable tissue, an energizing potential may be applied to the electrode portions thereof, such as to create an electric field to which the tissue at the target site is exposed. The energizing potential (and the resulting electric field) may be characterized by various parameters, such as, for example, frequency, amplitude, pulse width (duration of a pulse or pulse length). Suitable energy sources include electrical waveform generators. The energy source generates an electric field with desired characteristics for the treatment to be performed at the target site. For instance, the electric field may be generated to have suitable characteristic waveform output in terms of frequency, amplitude, pulse width, and polarity. The electric current flows between the electrodes and through the tissue proportionally to the potential (e.g., voltage) applied to the electrodes. The supplied electric current provided by the energy source may deliver a pulse sequence to the target site. For example, an energy source may supply various waveforms in one or more pulse sequences tailored to the desired application.

Energy-delivering assemblies, devices, systems, and methods described herein may be utilized for electroporation, irreversible electroporation (IRE), and/or electropermeabilization techniques to apply external electric fields (electric potentials) to cell membranes to significantly increase permeability of the plasma membrane of the cell, such as to improve uptake of therapeutic materials by the cell. Optionally, the energy applied to the cell may change the characteristics of the cell membranes (e.g., porosity), such as irreversibly, resulting in cell death (e.g., by apoptosis and/or necrosis). Such techniques may advantageously be used to treat/apply therapy without raising the temperature of the surrounding tissue to a level at which permanent damage may occur to the surrounding tissue, support structure, and/or regional vasculature Application of IRE pulses to cells may thus be an effective way for ablating large volumes of undesirable tissue with no or minimal detrimental thermal effects to the surrounding healthy tissue.

Various embodiments of electrode devices, assemblies, systems, and various associated methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

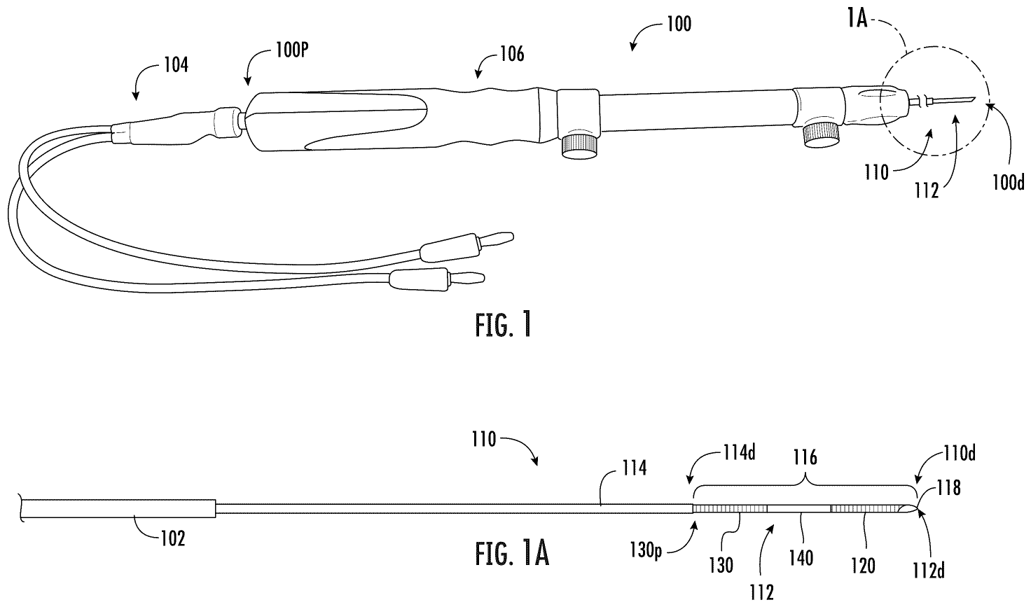

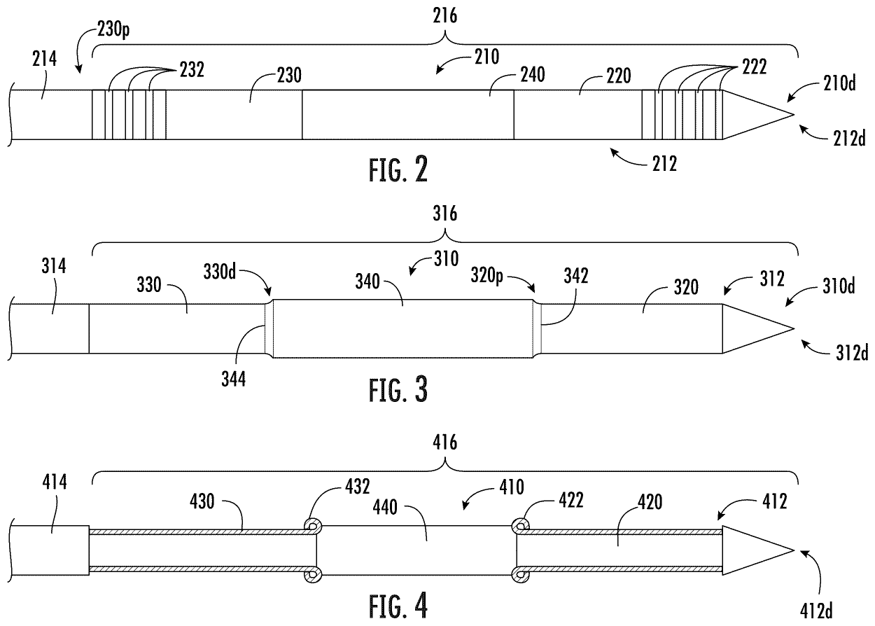

Turning now to the drawings, an example of an embodiment of an energy-delivering treatment system 100 is illustrated in FIG. 1. The energy-delivering treatment system 100 includes an energy-delivering assembly 110 extending along the distal end 100d of the energy-delivering treatment system 100. The energy-delivering assembly 110 includes an energy-delivering member 112 formed of an electrically-conductive material such as medical grade stainless steel, platinum, gold, nitinol, a cobalt-chromium alloy, a nickel-cobalt alloy such as MP35N, or other alloys, or materials plated with electrically-conductive materials, etc. An insulation member 114 is positioned around a proximal portion of the energy-delivering member, such as to restrict delivery of energy to an energy-delivering distal region 116 of the energy-delivering member 112, and/or to prevent delivery of energy to a patient along the insulated portion of the energy-delivering member 112 proximal to the energy-delivering distal region 116. As may be appreciated with reference to FIG. 2, the energy-delivering distal region 116 of the energy-delivering member 112 is a region extending from the distal tip or end 112d (e.g., free/terminal end) of the energy-delivering member 112 proximally to the distal end 114d of the insulation material 114 to define electrodes 120, 130 of the energy-delivering assembly 110 along the energy-delivering member 112, as described in further detail below. In some embodiments, the distal end 112d of the energy-delivering member 112 ends in a sharp distal tip 118. The sharp distal tip 118 may be configured to pierce (e.g., percutaneously) tissue/organs/tumor masses, such as in a manner known those of ordinary skill in the art. For instance, the energy-delivering member 112 may be in the form of a trocar. However, the present disclosure need not be limited to a particular configuration, and, more generally, need not be limited in this regard.

The energy-delivering treatment system 100 optionally includes a sheath 102, as may be appreciated with reference to FIG. 2. The energy-delivering assembly 110 may be deliverable to a target site with a sharp distal tip 118 thereof within the sheath 102. As such, the sheath 102 protects the passage through which the energy-delivering assembly 110 is extended (e.g., a working channel of an endoscope, a body lumen, etc.) from the sharp distal tip 118 of the energy-delivering member 112. The sheath 102 is selectively proximally retractable with respect to the energy-delivering member 112 and/or the energy-delivering member 112 is distally extendable with respect to the sheath 102 to expose at least the distal region 116 of the energy-delivering assembly 110 with respect to a target site during use of the energy-delivering assembly 110 for therapeutic/treatment purposes.

Optionally, a power source is coupled to the proximal end 100p of energy-delivering treatment system 100. The energy-delivering treatment system 100 may include a power connector 104, such as wiring configured to be coupled to an energy source such as known those of ordinary skill in the art and selectable by known means based on the type of energy to be applied by the energy-delivering treatment system 100. For instance, the energy source may be selected, in a manner known to those of ordinary skill in the art, to apply energy of a nature and in a manner to energize the energy-delivering assembly 110 to be used for electroporation and/or IRE. The present disclosure need not be limited by the details of the energy source.

The energy-delivering treatment system 100 optionally includes a handle 106 operatively coupled with the energy-delivering assembly 110, such as to control elements of the energy-delivering assembly 110. For instance, the handle 106 may be configured to control the relative positions of the sheath 102 and the energy-delivering assembly 110, and/or to control and/or adjust the position of the energy-delivering assembly 110 (e.g., the energy-delivering member 112 thereof). In some aspects, the energy-delivering assembly 110 is delivered to a target site within a patient through a delivery device, such as a sheath or an endoscope, having a lumen or working channel therethrough sized to allow passage of the energy-delivering assembly 110 and optional sheath 102 therethrough. Such delivery device may be selected from a variety of delivery devices known to those of ordinary skill in the art, the present disclosure not being limited in this regard. In some aspects, the handle 106 may be configured to control and/or adjust the position of the sheath 102 and/or the energy-delivering assembly 110 with respect to such delivery device.

In accordance with various principles of the present disclosure, the energy-delivering assembly 110 is an elongate flexible assembly capable of being navigated through a patient's body, such as through natural orifices and/or through tubular elongate members inserted into the patient's body. More particularly, the energy-delivering member 112 may be elongated and sufficiently flexible to be able to be inserted (e.g., transluminally) into the body and navigated through potentially tortuous pathways within the body, or at least being capable of bending or turning with/within natural, nonlinear anatomical structures. Additionally or alternatively, the energy-delivering member 112 may be sufficiently resilient so as not to break as it is being navigated. It will be appreciated that determination of appropriate length, flexibility, and/or resiliency of an energy-delivering member 112 used in accordance with various principles of the present disclosure may readily be determined by those of ordinary skill in the art, such as based on the material, size, shape, configuration, and/or dimensions of the energy-delivering member 112, the present disclosure not necessarily being limited to specific parameters.

In accordance with various principles of the present disclosure, the energy-delivering assembly 110 is configured as a bipolar probe, and defines therealong a first electrode 120, and a second electrode 130. In some aspects, the energy-delivering assembly 110 may be considered a linear bipolar probe with the electrodes 120, 130 formed along the same energy-delivering member 112 and generally colinear yet axially spaced apart from each other. In a linear configuration, the first electrode 120 may be referenced as a distal electrode 120, and the second electrode 130 may be referenced as a proximal electrode. It will be appreciated that references herein to “first”, “second”, “proximal”, and “distal” may be simply for the sake of convenience without intent to limit to particular orders or positions unless explicitly stated and/or required by a particular procedure, therapy, technique, etc. (such terms being used interchangeably herein without intent to limit unless specifically indicated).

In the example of an embodiment of an energy-delivering assembly 110 illustrated in FIG. 2, an electrode-defining insulation member 140 is provided along a selected extent (e.g., a limited axial extent) of the energy-delivering member 112 to insulate such extent of the energy-delivering member 112 and to prevent delivery of energy along such extent. In some aspects, the electrode-defining insulation member is positioned over and circumferentially around the energy-delivering member 112 to insulate the energy-delivering member 112 and to limit/prevent energy from being delivered by the energy-delivering member 112 through the electrode-defining insulation member 140. In the example of an embodiment of an energy-delivering assembly 110 illustrated in FIG. 2, the distal region 116 of the energy-delivering member 112 of the energy-delivering assembly 110 (distal to the distal end 114d of the insulation member 114) is configured to deliver energy for treatment/therapeutic purposes, and the electrode-defining insulation member 140 is positioned along an extent thereof to define a first electrode 120 spaced apart from a second electrode 130. As such, the example of an embodiment of an energy-delivering assembly 110 illustrated in FIG. 2 may be considered a bipolar energy-delivering assembly 110, which may alternatively be referenced as a bipolar probe. The above-described insulation member 114 extends over a proximal portion of the energy-delivering member 112, proximal to the proximal end 130p of the second, proximal electrode 130, to insulate the portion of the energy-delivering member 112 extending proximal to the second, proximal electrode 130. The insulation member 114 may thereby be considered to define the energy-delivering distal region 116 of the energy-delivering member 112 and/or to limit delivery of energy to a target site via the first, distal electrode 120 and the second, proximal electrode 130.

As appreciated by those of ordinary skill in the art, arcing is most likely to initiate at areas of high electrical field density, such as along the edges of electrode-to-insulation interfaces. A multi-electrode (e.g., bipolar) probe formed in accordance with various principles of the present disclosure has one or more modifications to the electrodes and/or the insulation member(s) thereof configured to reduce/minimize, and preferably to eliminate, arcing between the electrodes thereof.

In some aspects, a bipolar probe includes features increasing echogenicity, such as to enhance visibility with ultrasound visualization systems and/or method. Echogenic features may include radial grooves (or other features/coatings) configured to increase echogenicity. For instance, echogenic grooves may act to concentrate the local electric field (e.g., as an electric field concentration structure or region). In contrast with typical echogenic grooves, in the example of an embodiment of an energy-delivering assembly 210 illustrated in FIG. 2, echogenic features are formed in accordance with various principles of the present disclosure by being selectively placed along at least one of the electrodes 220, 230 at a location furthest from the other of the electrodes 220, 230. For instance, in the example of an embodiment of an energy-delivering assembly 210 illustrated in FIG. 2, one or more echogenic features 222 are provided along a distal end 220d of the first, distal electrode 220 (e.g., at a location along the first electrode 220 furthest from the second electrode 230) and/or one or more echogenic features 232 are provided along a proximal end 230p of the second, proximal electrode 230 (e.g., at a location along the second electrode 230 furthest from the first electrode 220) Such placement has been discovered to reduce/minimize the risk of arc formation. Without being bound by theory, such placement is believed to reduce stress concentrations near material transitions of the energy-delivering assembly 210 and thereby to reduce/minimize/eliminate arcing between the electrodes 220, 230. It will be appreciated that various features of the example of an embodiment of an energy-delivering assembly 210 illustrated in FIG. 2 may be similar to features of the example of an embodiment of an energy-delivering assembly 110 illustrated in FIG. 1 and FIG. 2, and are indicated with the same reference characters differing in value by a multiple of 100, reference being made to the above descriptions of similar elements and operations for the sake of brevity and convenience, and without intent to limit.

Additionally or alternatively, in accordance with various principles of the present disclosure the shape and/or configuration of the electrodes at the interface between the electrodes and at least the intermediate, electrode-defining insulation member between the electrodes may be modified to reduce/minimize/eliminate arcing between the electrodes. For instance, in some aspects, the interface between one or both electrodes of a bipolar probe and at least the intermediate, electrode-defining insulation member between such electrodes is configured to eliminate sharp corners. Without being bound by theory, reduction of sharp corners is believed to reducing concentration of energy at the interface/transition between an electrode and an adjacent insulation member, and thereby to reduce arcing between the electrodes.

In the example of an embodiment of an energy-delivering assembly 310 illustrated in FIG. 3, the interface between one or both electrodes 320, 330 and an electrode-defining insulation member 340 therebetween may be chamfered (e.g., sloped, angled, etc.) and/or filleted (e.g., rounded, curved, etc.). For instance, an electrode-defining insulation member 340 is typically positioned over and around (e.g., circumferentially around to cover) a portion of the energy-delivering member 312 to define separate electrodes 320, 330 along an energy-delivering distal region 316 of the energy-delivering member 312. In some aspects, the outer diameter of the electrode-defining insulation member 340 may be larger than the outer diameter of one or both of the electrodes 320, 330, although the reverse relative dimensions (with the outer diameter of the electrodes larger than the diameter of the insulation member), or all outer diameters being substantially the same, would be acceptable as well. In accordance with various principles of the present disclosure, one or both of the edges 342, 344 of the electrode-defining insulation member 340, and/or one or both of the proximal end 320p of the first, distal electrode 320 or the distal end 330d of the second, proximal electrode 330 along the interface of the electrode-defining insulation member 340 with the electrodes 330, 340, respectively, are chamfered, rounded, backfilled (potted), or filleted. Such configuration provides a more gradual transition between the outer diameter of the electrode-defining insulation member 340 and the outer diameter of one or both of the electrodes 320, 330 than in prior bipolar probes. It will be appreciated that the energy-delivering member 312 (forming the electrodes 320, 330) and/or the electrode-defining insulation member 340 and/or the proximal insulation member 314 may otherwise have similar material properties as the above-described energy-delivering member 112, electrode-defining insulation member 140, and/or proximal insulation member 114, reference accordingly being made to the above descriptions for the sake of brevity and without intent to limit. Moreover, it will be appreciated that various features of the example of an embodiment of an energy-delivering assembly 310 illustrated in FIG. 3 may be similar to features of the example of an embodiment of an energy-delivering assembly 110 illustrated in FIG. 1 and FIG. 2, and are indicated with the same reference characters differing in value by a multiple of 200, reference being made to the above descriptions of similar elements and operations for the sake of brevity and convenience, and without intent to limit

In the example of an embodiment of a bipolar energy-delivering assembly 410 illustrated in FIG. 4, a bipolar energy-delivering assembly 410 is formed similar to the above-described energy-delivering assemblies, with an energy-delivering member 412 defining a first electrode 420 spaced apart form a second electrode 430 by an electrode-defining insulation member 440 positioned therebetween. Accordingly, various features of the example of an embodiment of an energy-delivering assembly 410 illustrated in FIG. 4 may be similar to features of the example of an embodiment of an energy-delivering assembly 110 illustrated in FIG. 1 and FIG. 2, and are indicated with the same reference characters differing in value by a multiple of 300, reference being made to the above descriptions of similar elements and operations for the sake of brevity and convenience, and without intent to limit. In the example of an embodiment of an energy-delivering assembly 410 illustrated in FIG. 4, arcing is reduced between the electrodes 420, 430 (illustrated in cross-section to facilitate understanding of an example of an embodiment thereof) by providing/forming a rolled tube wall 422, 432 at the interface of one or both of the electrodes 420, 430 with the electrode-defining insulation member 440 therebetween. Without being bound by theory, the curvature of the electrically conductive material of the energy-delivering member 412, from which the one or both electrodes 420, 430 are formed, along the interface with the electrode-defining insulation member 440, reduces concentration of energy which may otherwise cause arcing between the electrodes 420, 430. Even if there is a generally sharp material transition between the electrode-defining insulation member 440 and one or both of the electrodes 420, 430, without being bound by theory, it is believed that the removal of square corners at the insulation interface is still beneficial in reducing arcing at such interfaces.

In addition to or instead of modifying various configurations and/or properties of the electrically-conductive regions of a bipolar probe to reduce arcing between the electrodes, various modifications may be made in accordance with various principles of the present disclosure to the insulation members thereof. As may be appreciated, one or more insulation members may be considered to define the electrodes of an energy-delivering assembly by covering portions of an electrically-conductive energy-delivering member to limit energy delivery to selected areas left exposed (not covered) by the insulation member. In accordance with various principles of the present disclosure, various modifications may be made to configurations and/or materials of the insulation members of an energy-delivering assembly to reduce arcing.

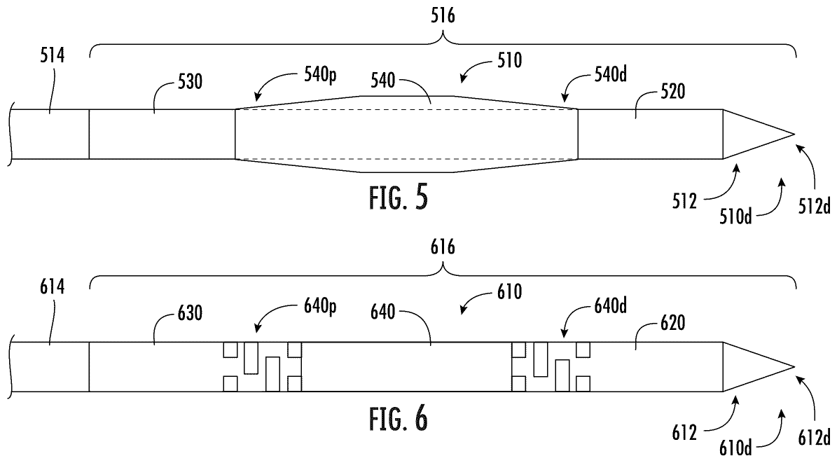

In the example of an embodiment of an energy-delivering assembly 510 illustrated in FIG. 5, a proximal insulation member 514 and an electrode-defining insulation member 540 are provided over an electrically-conductive energy-delivering member 512 to define a first electrode 520 and a second electrode 530, such as in a manner similar to that described with respect to the example of an embodiment of an energy-delivering assembly 110 illustrated in FIG. 1 and FIG. 2. However, as described in further detail below, the electrode-defining insulation member 540 is modified to reduce/minimize/eliminate arcing between the electrodes 520, 530. Generally, similar to the energy-delivering assembly 110 illustrated in FIG. 2, the electrodes 520, 530 are defined along an energy-delivering distal region 516 of the energy-delivering member 512 defined by a distal portion of an electrically-conductive energy-delivering member 512 left uncovered by an insulation member 514 covering a proximal portion of the energy-delivering member 512. An additional insulation member 540 is positioned over an intermediate region of the energy-delivering distal region 516 to define the electrodes 520, 530 of the bipolar energy-delivering assembly 510. It will be appreciated that various features of the example of an embodiment of an energy-delivering assembly 510 illustrated in FIG. 5 may be similar to features of the example of an embodiment of an energy-delivering assembly 110 illustrated in FIG. 1 and FIG. 2, and are indicated with the same reference characters differing in value by a multiple of 400, reference being made to the above descriptions of similar elements and operations for the sake of brevity and convenience, and without intent to limit.

In accordance with various principles of the present disclosure, the electrode-defining insulation member 540 of the example of an embodiment of an energy-delivering assembly 510 illustrated in FIG. 5 is modified compared to the electrode-defining insulation member 140 of the example of an embodiment of an energy-delivering assembly 110 illustrated in FIG. 2 by a more gradual transition of the electrode-defining insulation member 540 with respect to the underlying energy-delivering member 512. More particularly, as may be appreciated with reference to the example of an embodiment illustrated in FIG. 5, the electrode-defining insulation member 540 is positioned over (e.g., on top of and circumferentially around, such as to cover) the energy-delivering member 512 of the energy-delivering assembly 510. The illustrated example of an embodiment of an electrode-defining insulation member 540 is configured such that the transition from the insulative material of the electrode-defining insulation member 540 to the electrically-conductive material of the energy-delivering member 512 is gradual geometrically (e.g., no sharp transitions which may cause arcing). For instance, the electrode-defining insulation member 540 may start at one or both of the distal end 540d or proximal end 540p thereof as a very thin initial layer of insulation, gradually increasing in thickness towards a center region of the electrode-defining insulation member 540, such as may be understood by to those of ordinary skill in the art in tapering of members such as the insulation member 540 at edges thereof. Partial electrical conductivity, from the underlying energy-delivering member 512, may occur through the thinner ends of the insulation member 540, gradually decreasing with the increasing thickness and resistance of the insulation member 540. Without being bound by theory, it is believed that the gradual increase in thickness of the electrode-defining insulation member 540 over the underlying energy-delivering member 512 may help to mitigate the transition between conductor and insulator, and thereby to reduce/minimize/eliminate arcing between the electrodes 520, 530 separated by the electrode-defining insulation member 540. It may be appreciated that such taper may also present atraumatic surfaces during deliver and insertion of the energy-delivering assembly 510 with respect to tissue.

Another manner of modifying properties and/or characteristics of an electrode-defining insulation member of an energy-delivering assembly formed in accordance with various principles of the present disclosure is illustrated with reference to FIG. 6. Similar to the example of an embodiment of an energy-delivering assembly 110 illustrated in FIG. 1 and FIG. 2, the example of an embodiment of an energy-delivering assembly 610 illustrated in FIG. 6 has an electrode-defining insulation member 640 provided over a distal region of an electrically-conductive energy-delivering member 612 to define a first electrode 620 on one side of the electrode-defining insulation member 640 and a second electrode 630 on the other side of the electrode-defining insulation member 640. It will be appreciated that various features of the example of an embodiment of an energy-delivering assembly 610 illustrated in FIG. 6 may be similar to features of the example of an embodiment of an energy-delivering assembly 110 illustrated in FIG. 1 and FIG. 2, and are indicated with the same reference characters differing in value by a multiple of 500, reference being made to the above descriptions of similar elements and operations for the sake of brevity and convenience, and without intent to limit.

In contrast with the electrode-defining insulation member 140 of the example of an embodiment of an energy-delivering assembly 110 illustrated in FIG. 1, the electrode-defining insulation member 640 of the example of an embodiment of an energy-delivering assembly 640 illustrated in FIG. 6 is configured to define a more gradual transition between the electrically-conductive electrodes 620, 630 on either side of the electrode-defining insulation member 640. In particular, the electrode-defining insulation member 640 provided over the electrically-conductive energy-delivering member 612 of the energy-delivering assembly 610 is discontinuous along one or both of the distal end 640d (adjacent the first, distal electrode 620) or the proximal end 640p (adjacent the second, proximal electrode 630) of the electrode-defining insulation member 640. Without being bound by theory, such configuration is believed to break up electric field concentrations along the ends 640d, 640p of the electrode-defining insulation member 640, and thereby to reduce/minimize/eliminate arcing between the electrodes 620, 630. For instance, the slots may spread electric field concentrations from a single line at the interface between the electrode-defining insulation member 640 and the electrodes 620, 630, to a broader/wider area. In the example of an embodiment of an energy-delivering assembly 610 illustrated in FIG. 6, the electrode-defining insulation member 640 is selectively slotted to define areas of discontinuity therealong to reduce/minimize/eliminate arcing between the electrodes 620, 630 thereof. For instance, the ends 640d, 640p of the electrode-defining insulation member 640 may be a coating/covering over the electrodes 620, 630 (e.g., masking) but could also be integrated into the design of the electrodes themselves. (i.e., a hypotube with a laser-cutout pattern). It will be appreciated that the pattern/configuration of discontinuities along the electrode-defining insulation member 640 may be other than slotted (e.g., circular, curved, ovoid, etc.), the present disclosure not being limited in this regard.

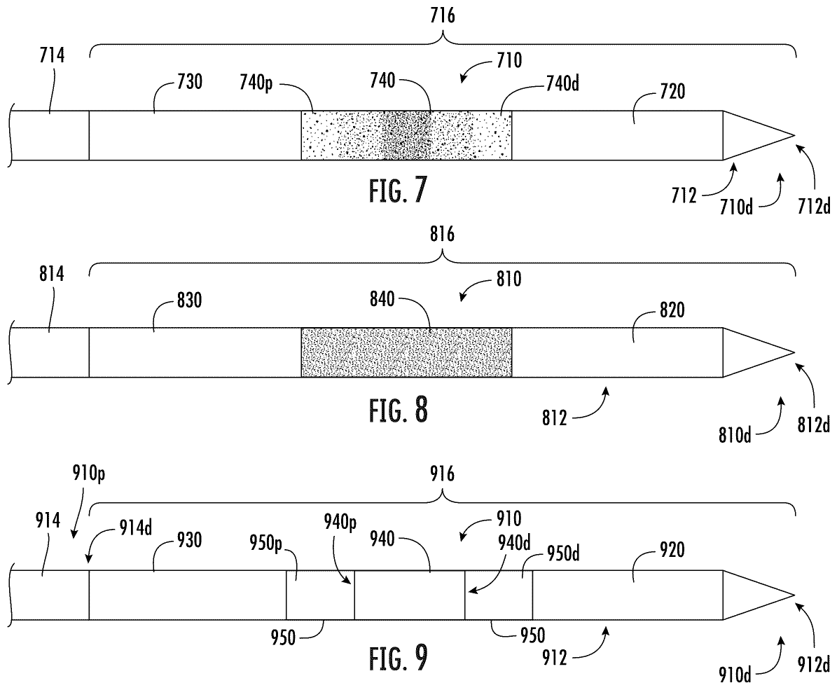

In accordance with various principles of the present disclosure, an additional or alternative approach to reducing arcing between electrodes may be achieved by doping selected regions of the electrode-defining insulation member defining and separating electrodes along an energy-delivering member of an energy-delivering assembly. The doping may occur as a gradient, to increase conductivity of the insulation member near the interfacing edges adjacent the electrodes, or across the entire electrode-defining insulation member (or reducing the conductivity of the electrode) to increase the overall conductivity (but not approaching tissue conductivity). For instance, in the example of an embodiment of an energy-delivering assembly 710 illustrated in FIG. 7, a doped electrode-defining insulation member 740 is positioned over a portion of a distal region of the energy-delivering member 712 to define electrodes 720, 730 of an energy-delivering distal region 716 of the energy-delivering assembly 710. The doping of the electrode-defining insulation member 740 illustrated in FIG. 7 may be gradual (e.g., in a gradient, gradually increasing in a direction towards the distal end 740d and/or the proximal end 740p thereof and towards an adjacent electrode 720, 730) so that the electrode-defining insulation member 740 has a degree of electrical conductivity at the transition along the distal end 740d thereof with respect to the first, distal electrode 720 and/or along the proximal end 740p thereof with respect to the second, proximal electrode 730. Alternatively, in the example of an embodiment of an energy-delivering assembly 810 illustrated in FIG. 8, an electrode-defining insulation member 840 separating the electrodes 820, 830 of an energy-delivering distal region 816 of the energy-delivering assembly 810 may be substantially uniformly doped along its entire length to be slightly more conductive, thereby reducing sharp changes in electrical conductivity between the electrodes 820, 830. Without being bound by theory, a more gradual transition in electrical conductivity between electrodes of a bipolar probe and the insulation therebetween may be achieved by masking, coatings, coverings, etc., to alter conductivity across the transition areas and thereby to reduce arcing at such transition areas (relative to prior bipolar probes). Alternatively or additionally, energy may be correlated to current density/concentration. It will be appreciated that various features of the examples of embodiments of energy-delivering assemblies 710, 810 illustrated in FIG. 7 and FIG. 8 may be similar to features of the example of an embodiment of an energy-delivering assembly 110 illustrated in FIG. 1 and FIG. 2, and are indicated with the same reference characters differing in value by a multiple of 600 (in the case of the energy-delivering assembly 810) or 700 (in the case of the energy-delivering assembly 810), reference being made to the above descriptions of similar elements and operations for the sake of brevity and convenience, and without intent to limit.

In addition to or instead of modifying electrodes and/or insulation members of a bipolar probe to reduce arcing, a variety of materials may be added to a bipolar energy-delivering assembly formed in accordance with various principles of the present disclosure to reduce arcing. More particularly, in accordance with various principles of the present disclosure, a third (or possibly more) material(s) acting as a buffer/matching layer may be added at transitions between materials of different electrical conductivities to decrease the disparity between the electrical conductivities at the transition therebetween. Without being bound by theory, more gradual changes in electrical conductivity are believed to reduce likelihood of arcing between spaced apart electrodes and/or electrically conductive members in general.

For instance, in the example of an embodiment of a bipolar energy-delivering assembly 910 illustrated in FIG. 9, an additional transition member 950 may be provided over the electrically-conductive energy-delivering member 912 of the energy-delivering assembly 910 adjacent one or both sides/ends of the electrode-defining insulation member 940 defining and spacing apart the electrodes 930, 940 of the bipolar energy-delivering assembly 910. More particularly, like the example of an embodiment of an energy-delivering assembly 110 described above with reference to FIG. 2, the example of an embodiment of an energy-delivering assembly 910 illustrated in FIG. 0 includes an electrically-conductive energy-delivering member 912 with an insulation member 914 positioned around a proximal portion 910p thereof to define an energy-delivering distal region 916 of the energy-delivering assembly 910 along which at least portions of the electrically-conductive energy-delivering member 912 are exposed to define electrodes 920, 930 of the energy-delivering assembly 010. An electrode-defining insulation member 940 is positioned over the energy-delivering member 912 along an intermediate portion of the energy-delivering region 916 (between the distal end 912d of the energy-delivering member 912 and the distal end 914d of the insulation member 914) to differentiate regions or portions (e.g., to differentiate electrically conductive regions or portions) of the energy-delivering member 912 to define a first electrode 920 spaced apart from a second electrode 930. In the illustrated example of an embodiment, in accordance with various principles of the present disclosure, a distal transition member 950d is provided between the first, distal electrode 920 and the distal end 940d electrode-defining insulation member 940, and/or a second transition member 950p is provided between the second, proximal electrode 930 and the proximal end 940p of the electrode-defining insulation member 940. It will be appreciated that various other features of the example of an embodiment of an energy-delivering assembly 910 illustrated in FIG. 9 may be similar to features of the example of an embodiment of an energy-delivering assembly 110 illustrated in FIG. 1 and FIG. 2, and are indicated with the same reference characters differing in value by a multiple of 800, reference being made to the above descriptions of similar elements and operations for the sake of brevity and convenience, and without intent to limit.

In accordance with various principles of the present disclosure, one or more transition members 950 are positioned along (e.g., over and circumferentially around, such as to cover) the energy-delivering member 912 of the example of an embodiment of an energy-delivering assembly 910 illustrated in FIG. 910 between the electrically conductive portions of the energy-delivering member 912 (defining the electrodes 920, 930) and the electrode-defining insulation member 940 therebetween. The one or more transition members 950 are configured as a conductive material matching layer/member and/or buffer between the electrically conductive regions (e.g., the electrodes 920, 930) of the energy-delivering distal region 916 of the energy-delivering assembly 910, and the electrode-defining insulation member 940. For instance, the transition member 950 may have an electrical conductivity which is lower than that of the electrodes 920, 930 to decrease the disparity of conductivity between the electrically-conductive electrodes 920, 930 and the electrode-defining insulation member 940 therebetween. The transition member 950 may include, without limitation, an additional different material, a material treatment, and/or other transition region between an insulation member and the electrodes it separates. The transition member 950 is selected to reduce arcing between the electrodes 920, 930 of the energy-delivering assembly 910 by having an intermediate electrical conductivity less than that of an adjacent electrode 920, 930, yet greater than the electrode-defining insulation member 940 (i.e., non-insulating/not nonconductive). The material of the transition member 950 may be an electrically-conductive, preferably biocompatible material (e.g., metal such as bulk metallic glass, nickel, chromium, tungsten, iron; alloys such as nitinol, nichrome, titanium) having an electrical conductivity which is less than that of the energy-delivering member 912; a low strength dielectric, preferably biocompatible, material (silicone, polypropylene, polyethylene, polycarbonate, polyether block amide. thermoplastic urethane (TPU), urethane, expanded PTFE (ePTFE)). having an electrical conductivity less than that of the energy-delivering member 910; or any material having an electrical conductivity level between that of the electrode-defining insulation member 940 and one or both electrodes 920, 930. Additionally or alternatively, a section of the energy-delivering member 912 closest to an electrode-defining insulation member 940 defining electrodes 920, 930 along the energy-delivering member 912 (e.g., a portion of one of the electrodes 920, 930 closer to the other of the electrodes 920, 930) may be treated (precipitated, hardened, etc.) in such a manner that the electrical conductivity is reduced in that section. Such sections may be along the general region of the energy-delivering member 912 along which a separate transition member 950 may be positioned and thus are not separately indicated.

In addition to or instead of one or more of the above features, members, treatments, etc., which reduce arcing between electrodes of a bipolar probe in accordance with various principles of the present disclosure, an additional material may be provided over a region of the electrically conductive material forming at least one electrode of the probe to reduce the electrical conductivity in such region. For instance, in addition to or instead of altering a base material such as described above, an additional layer of material may be provided over the energy-delivering member of an energy-delivering assembly formed in accordance with various principles of the present disclosure to alter the surface properties thereof to reduce/minimize/eliminate arcing between electrodes thereof. Reference may be made herein to a layer, coating, etc., interchangeably without intent to limit. The coating may be formed of a material which is less electrically-conductive than the material of the energy-delivering member over which the coating is applied. Examples of acceptable materials for such coatings include, without limitation, an oxide, ceramic, silicone, polypropylene, polyethylene, polycarbonate, polyether block amide. TPU, urethane, ePTFE, patterned, etc., preferably biocompatible, material.

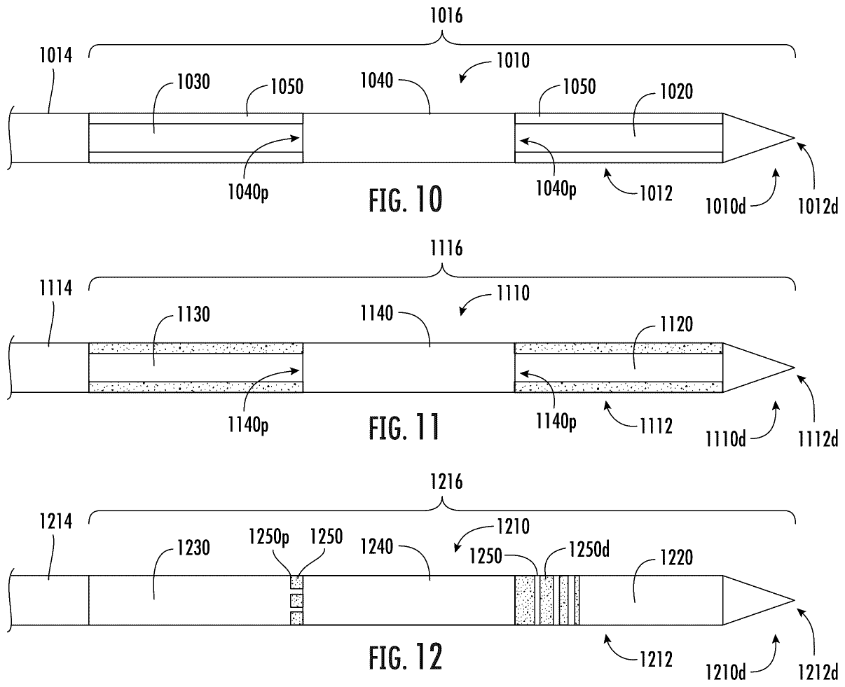

A coating or layer may be provided in a variety of manners to reduce/minimize/eliminate arcing in accordance with various principles of the present disclosure. For example, in the example of an embodiment of an energy-delivering assembly 1000 illustrated in FIG. 10, an oxide layer which may be applied (e.g., by application of heat, chemical, atmospheric conditions and/or combination thereof) over the energy-delivering member 1012 of the energy-delivering assembly 1010 may be modified to reduce/minimize/eliminate arcing between electrodes 1020, 1030 of the energy-delivering assembly 1010. More particularly, similar to above-described examples of embodiments of energy-delivering assemblies, an energy-delivering distal region 1016 of an energy-delivering assembly 1010 may be defined along a distal region of an electrically-conductive energy-delivering member 1012 distal to an insulation member 1014 covering a proximal portion of the energy-delivering member 1012. An electrode-defining insulation member 1040 may be provided along an intermediate region of the energy-delivering distal region 1016 to define and separate electrodes 1020, 1030 along the energy-delivering distal region 1016. It will be appreciated that various other features of the example of an embodiment of an energy-delivering assembly 1010 illustrated in FIG. 10 may be similar to features of the example of an embodiment of an energy-delivering assembly 110 illustrated in FIG. 1 and FIG. 2, and are indicated with the same reference characters differing in value by a multiple of 900, reference being made to the above descriptions of similar elements and operations for the sake of brevity and convenience, and without intent to limit. In accordance with various principles of the present disclosure, an oxide layer over the electrodes 1020, 1030 is increased in thickness (relative to a typical oxide layer), such as by passivation, anodization, etc., of the electrically conductive energy-delivering member 1012. Such increase in thickness is at least in a region closer to the electrode-defining insulation member 1040 (e.g., the distal end 1040d and/or the proximal end 1040p of the insulation member 1040 adjacent an electrode 1020 and/or 1030). Without being bound by theory, a thicker oxide coating may create a gradual transition from the electrical conductivity of one or both of the electrodes 1020, 1030 to the nonconductive electrode-defining insulation member 1040 between the electrodes 1020, 1030.

Additionally or alternatively, at least a portion of the electrodes of a bipolar energy-delivering assembly formed in accordance with various principles of the present disclosure may be coated with a less conductive material or a nonconductive coating. For instance, the electrodes 1120, 1130 of the example of an embodiment of an energy-delivering assembly 1110 illustrated in FIG. 11 may be coated with an a less conductive material or a nonconductive coating, at least adjacent to an electrode-defining insulation member 1140 defining and separating the electrodes 1120, 1130 along the energy-delivering member 1112 defining the energy-delivering portion of the energy-delivering assembly 1110. It will be appreciated that various features of the example of an embodiment of an energy-delivering assembly 1110 illustrated in FIG. 11 may be similar to features of the example of an embodiment of an energy-delivering assembly 110 illustrated in FIG. 1 and FIG. 2, and are indicated with the same reference characters differing in value by a multiple of 1000, reference being made to the above descriptions of similar elements and operations for the sake of brevity and convenience, and without intent to limit. In accordance with various principles of the present disclosure, a coating 1150 is applied over at least a portion of at least one of the electrodes 1120, 1130, such as adjacent to the electrode-defining insulation member 1140 to define a less conductive region along the at least one of the electrodes 1120, 1130. The coating may made of a material different from that of the energy-delivering member 1112, such as, without limitation, a bulk metallic glass, ceramic, polymers, doped polymers, etc. Without being bound by theory, such coating may create a gradual transition from the electrical conductivity of one or both of the electrodes 1120, 1130 to the nonconductive electrode-defining insulation member 1140 between the electrodes 1120, 1130.

In some aspects, an oxidized layer and/or an applied coating as described above with reference to the examples of embodiments of energy-delivering assemblies 1010, 1110 illustrated, respectively in FIG. 10 and FIG. 11, may be patterned (e.g., have discontinuities) using masking and/or other application techniques such as known those of ordinary skill in the art. The pattern may be selected to manipulate the electric fields between the electrodes of the energy-delivering assembly to reduce arcing therebetween. For instance, in the example of an embodiment of an energy-delivering assembly 1210 illustrated in FIG. 12, a coating 1250 over an energy-delivering member 1212 of an energy-delivering assembly 1210 may include a distal coating 1250d between the first, distal electrode 1320 and the electrode-defining insulation member 1340, and/or a proximal coating 1250p between the second, proximal electrode 1230 and the electrode-defining insulation member 1240. It will be appreciated that various features of the example of an embodiment of an energy-delivering assembly 1210 illustrated in FIG. 12 may be similar to features of the example of an embodiment of an energy-delivering assembly 110 illustrated in FIG. 1 and FIG. 2, and are indicated with the same reference characters differing in value by a multiple of 1100, reference being made to the above descriptions of similar elements and operations for the sake of brevity and convenience, and without intent to limit. In the example of an embodiment of patterned coatings 1250d, 1250p illustrated in FIG. 12, the pattern may include axial spacings of coating (e.g., axial gaps between circumferentially extending coatings spaced apart axially from one another), such as the illustrated distal coating 1250d, and/or circumferential gaps along the coating as in illustrated proximal coating 1250p. The gaps need not be linear, but, instead, may have other shapes, patterns, topography, such as arcuate or otherwise. Without being bound by theory, patterns with gaps defined in a coating provided over the electrically-conductive energy-delivering member 1212 of an energy-delivering assembly 1210 such as illustrated in FIG. 12 may create a gradual transition from the electrical conductivity of one or both of the electrodes 1220, 1230 to the nonconductive electrode-defining insulation member 1240 between the electrodes 1220, 1230.

It will be appreciated that any of the above-described bipolar probes may be configured as linear probes, with the electrodes thereof axially spaced apart from each other. However, it will also be appreciated that configurations other than linear are within the scope and spirit of the present disclosure as well. Furthermore, although the above-described examples of embodiments are illustrated with only two electrodes, the above-described principles may be applied to energy-delivering assemblies with more than two electrodes, such as to other bipolar, unipolar, and/or monopolar devices. And, principles of the present disclosure may be applied to energy other than electroporation and/or IRE energy, such as other energy sources for ablation or otherwise. Finally, it will be appreciated that although not explicitly discussed, the distal end of any of the above-described energy-delivering members may end in a sharp distal tip (e.g., the energy-delivering member may be in the form of a trocar) or may have another configuration (e.g., atraumatic, such as blunt or soft or otherwise, such as a luminal device) as necessary or prescribed for the procedure to be performed with the energy-delivering assembly (e.g., may be a needle with a lumen therethrough). It will be appreciated that the devices, systems, assemblies, and methods disclosed herein may be delivered endoscopically, transluminally, or percutaneously, as well as used within other access devices such as other steerable luminal access devices.

It will be appreciated that all structures, devices, systems, assemblies, and methods discussed herein are examples implemented in accordance with one or more principles of this disclosure, and are not the only way to implement these principles, and thus are not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, assemblies, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An energy-delivering assembly comprising:
an energy-delivering member formed of an electrically-conductive material;
an electrode-defining insulation member positioned over a portion of said energy-delivering member to define a first electrode spaced apart from a second electrode by said electrode-defining insulation member; and
at least one arc-reducing interface between said electrode-defining insulation member and an adjacent one of said first and second electrodes.

2. The energy-delivering assembly of claim 1, wherein said arc-reducing interface comprises a region comprises a modified shape of at least one of said electrode-defining insulation member or said adjacent one of said first and second electrodes.

3. The energy-delivering assembly of claim 2, wherein said arc-reducing interface comprises a chamfer or fillet along an interface between said electrode-defining insulation member and said adjacent one of said first and second electrodes.

4. The energy-delivering assembly of claim 2, wherein said arc-reducing interface comprises a rolled wall of said first electrode adjacent said electrode-defining insulation member.

5. The energy-delivering assembly of claim 2, wherein said arc-reducing interface comprises a reduction in thickness in said electrode-defining insulation member adjacent to said adjacent one of said first and second electrodes relative to an intermediate region of said electrode-defining insulation member spaced apart from said adjacent one of said first and second electrodes.

6. The energy-delivering assembly of claim 2, wherein said arc-reducing interface comprises discontinuities in said electrode-defining insulation member at least adjacent to said adjacent one of said first and second electrodes.

7. The energy-delivering assembly of claim 6, wherein said electrode-defining insulation member is slotted to define the discontinuities therealong.

8. The energy-delivering assembly of claim 1, wherein:
at least one of said first electrode and said second electrode has an end adjacent said electrode-defining insulation member and an end spaced away from said electrode-defining insulation member;
echogenic features are provided closer to the end of said at least one of said first electrode and said second electrode spaced away from said electrode-defining insulation member than the end of said at least one of said first electrode and said second electrode adjacent said electrode-defining insulation member; and
the end of said at least one of said first electrode and said second electrode adjacent said electrode-defining insulation member and without echogenic features defines said arc-reducing interface.

9. The energy-delivering assembly of claim 1, wherein said arc-reducing interface is formed by doping at least a region of said electrode-defining insulation member to create a gradient in electrical conductivity between said electrode-defining insulation member and said adjacent one of said first and second electrodes.

10. The energy-delivering assembly of claim 1, wherein said arc-reducing interface comprises a non-insulating additional member between said electrode-defining insulation member and said adjacent one of said first and second electrodes, and having an electrical conductivity less than that of said adjacent one of said first and second electrodes.

11. The energy-delivering assembly of claim 1, wherein said arc-reducing interface comprises a coating over said adjacent one of said first and second electrodes.

12. The energy-delivering assembly of claim 1, wherein said first electrode and said second electrode are colinear to define said energy-delivering assembly as a bipolar linear probe.

13. An energy-delivering treatment system comprising:
an energy-delivering assembly comprising:
an energy-delivering member formed of an electrically-conductive material;
an electrode-defining insulation member positioned over a portion of said energy-delivering member to define a first electrode spaced apart from a second electrode by said electrode-defining insulation member; and
at least one arc-reducing interface between said electrode-defining insulation member and an adjacent one of said first and second electrodes; and
a power connector configured to deliver energy to said energy-delivering assembly to deliver energy to said energy-delivering member to deliver energy along said electrodes thereof.

14. The energy-delivering treatment system of claim 13, wherein said arc-reducing interface comprises a region comprises a modified shape of at least one of said electrode-defining insulation member or said adjacent one of said first and second electrodes.

15. The energy-delivering assembly of claim 13, wherein:
at least one of said first electrode and said second electrode has an end adjacent said electrode-defining insulation member and an end spaced away from said electrode-defining insulation member;
echogenic features are provided closer to the end of said at least one of said first electrode and said second electrode spaced away from said electrode-defining insulation member than the end of said at least one of said first electrode and said second electrode adjacent said electrode-defining insulation member; and
the end of said at least one of said first electrode and said second electrode adjacent said electrode-defining insulation member and without echogenic features defines said arc-reducing interface.

16. The energy-delivering assembly of claim 13, wherein said arc-reducing interface is formed by doping at least a region of said electrode-defining insulation member to create a gradient in electrical conductivity between said electrode-defining insulation member and said adjacent one of said first and second electrodes.

17. The energy-delivering assembly of claim 13, wherein said arc-reducing interface comprises a non-insulating additional member between said electrode-defining insulation member and said adjacent one of said first and second electrodes, and having an electrical conductivity less than that of said adjacent one of said first and second electrodes.

18. The energy-delivering assembly of claim 13, wherein said arc-reducing interface comprises a coating over said adjacent one of said first and second electrodes.

19. A method of applying electroporation or irreversible electroporation energy with a multi-electrode energy-delivering treatment system having at least a first electrode and a second electrode defined along an energy-delivering member of the multi-electrode energy-delivering treatment system by an electrode-defining insulation member positioned over the energy-delivering member to define and space apart the first electrode and the second electrode from each other, said method comprising:
delivering energy from an energy source to the energy-delivering member of the multi-electrode energy-delivering treatment system to deliver electroporation or irreversible electroporation energy to the first electrode and the second electrode of the multi-electrode energy-delivering treatment system; and
creating a gradient in electrical conductivity between the electrode-defining insulation member and at least one of the first electrode or the second electrode of the multi-electrode energy-delivering treatment system to reduce arcing between the first electrode and the second electrode thereof.

20.

21. The method of claim 19, wherein creating a gradient in electrical conductivity comprises at least one of: modifying features of at least one of the first electrode or the second electrode of the multi-electrode energy-delivering system; modifying features of the electrode-defining insulation member; adding a non-insulating member between the electrode-defining insulation member and at least one of the first electrode or the second electrode, the non-insulating member having an intermediate electrical conductivity less than the electrical conductivity of the least one of the first electrode or the second electrode; or coating at least a portion of at least one of the first electrode and the second electrode.
